(19) United States
(12) Patent Application Publication
(10) Pub. No.: US 2004/0127905 A1
(43) Pub. Date: Jul. 1, 2004

(54) Bone marrow multi-tap

(76) Inventors: Chu Sing Lim, Singapore (SG); Cheng Peng Teo, Singapore (SG); Denis Christopher Ngian Kwong Nyam, Singapore (SG)

Correspondence Address: Dennis G. LaPointe, Esq., Mason Law, P.A., Suite 500, 17757 U.S. Hwy. 19 N., Clearwater, FL, 33764, US

(21) Appl. No.: 10/411,983

(22) Filed: Apr. 11, 2003

(30) Foreign Application Priority Data
Dec. 31, 2002 (SG) 200207881-4

(51) Int. Cl. A61B 17/56

(52) U.S. Cl. 606/72

(57) ABSTRACT

A bone marrow multi-tap, which allows repeated access to bone marrow without repeated boring through the bone each time access is desired and which will reduce discomfort and pain to patients, is a rigid tube reversibly implanted by screw threads and anchored by lock nuts and washers into the bone. Each time access is required, the tube is uncapped and an interior sealing plug is removed. Bone marrow may then be extracted as needed and the multi-tap re-plugged and re-sealed. Alternatively, the plug and cap may be of a design such that a needle may be inserted into the bore of the multi-tap without the need to uncap and remove the plug. Substances, such as anti-coagulants or antibiotics, may be incorporated into the multi-tap as needed to obtain a desired medical outcome.

### FIELD OF THE INVENTION

The present invention relates to a means of accessing the interior of bones for the contents, particularly bone marrow, for medical or veterinary purposes.

In particular, this invention relates to a rigid, tube-like device that can be temporarily or permanently affixed to bone or cartilage tissue to allow repeated access to bone marrow for medical examination, diagnostic and therapeutic purposes, while minimizing pain and discomfort to the patient.

### BACKGROUND OF THE INVENTION

At present, bone marrow examinations for humans are performed to examine the blood cells found in bone marrow for abnormalities. These abnormalities are essential for the diagnosis of blood and bone marrow disorders. These disorders include cancers such as leukemias, myelodysplastic syndromes, multiple myeloma, certain lymphomas and non-cancerous disorders such aplastic anemia, pure red cell aplasia, cyclical neutropenia and others.

In a bone marrow examination, marrow is usually extracted from the interior of the hip or pelvic bone at the site known as the iliac crest. The site is first numbed with a local anesthetic before a needle is used to penetrate the underlying fat layers to the bone.

A hole is then made into the bone with the needle to penetrate hard cortical bone tissue through to the spongy interior of the bone before a small sample of marrow is removed by scraping. Collection of bone marrow may also be done by suction with a syringe in the technique known as aspiration.

This marrow examination procedure may cause some pain and discomfort to the patient even with the use of the local anesthetic. To track the progress of treatment for some diseases such as leukemia, repeated sampling at monthly intervals for some diseases may have to be performed.

This painful, repeated sampling causes distress to patients, with the need to make fresh punctures into bone at different sites to draw marrow samples. Patients, especially children with leukemia, may have to endure months of treatment and as many as six examinations.

Therefore, a need clearly exists for means to reduce the discomfort and pain experienced by the patient by eliminating the need to puncture the skin, subcutaneous fat and bone in repeated bone marrow examinations.

### SUMMARY OF THE INVENTION

The present invention seeks to provide a means to allow repeated examinations of bone marrow through a device affixed onto bone or cartilage tissue while minimizing pain and discomfort to the patient.

In particular, the device is a rigid tube screwed into a hole drilled in bone and further with lock nut sets. The bore of the device sealed with a plug and then covered with a removable cap. A hypodermic needle or biopsy needle may then be used to extract small quantities of bone marrow.

The device may nor may not protrude through the subcutaneous fat of the patient to the exterior as determined by the surgeon, depending on the medical circumstances of the patient.

### BRIEF DESCRIPTION OF THE FIGURES

The present invention will now be briefly described, by way of example, with reference to the following figures. The figures are meant to be exemplary only and should not be interpreted to be limiting in any way. A person skilled in the art will appreciate that variations may be made to the invention without departing from the scope and spirit of the invention.

FIG. 1 illustrates two possible sites such as the breast bone (sternum) or hip (pelvis) where the bone marrow device may be implanted.

FIG. 2 shows the main elements of one embodiment of the invention.

FIG. 3 is one embodiment of the invention where the cap protrudes above the level of the skin.

FIG. 4 is another embodiment of the invention which does not protrude from the surface of the skin.

FIG. 5 is yet another embodiment of the invention which uses one or more washers with dampening capabilities under a flared base to obviate the need for separate lock nut sets to anchor the invention to bone.

### DETAILED DESCRIPTION OF THE DRAWINGS

A preferred embodiment of the invention will now be described. It shall be apparent to one skilled in the art that the invention may be practiced with variations in the lesser details of the invention, the fabrication and implantation methods, as well as choice of materials.

Some of these details may not be described at length so as not to obscure the invention.

While the specifications of the present invention are described mainly for use in humans, use in animals is not excluded and is under the scope and spirit of this invention.

Current medical practice is to make punctures of the fat and bone, usually at the hip bone or the breast bone, to extract bone marrow for examination to track the progression of certain disease so as to determine the efficacy of treatment. Such procedures are painful and uncomfortable to the patient.

While one invention, U.S. Pat. No. 5,120,312 Method and Apparatus for Catheterization, permits access to the interior of bones, it is neither structurally ideal nor optimized for this present purpose of extracting bone marrow. In addition, U.S. Pat. No. 5,120,312 neither teaches nor anticipates the present invention.

Hence there is a clear need to meet the specific application of repeated access to bone marrow for extraction and examination.

Differences will be drawn between U.S. Pat. No. 5,120,312 and the present invention to distinguish between the two.

The present invention is a device, a bone marrow tap, for directly accessing the interior of bones, particularly the larger bones of the body, to allow repeated examinations or sampling of bone marrow to be performed with minimal pain and discomfort to the patient.

Depending on the medical circumstances, the device may or may not protrude from the surface of the skin.

The noun terms, “device” or “tap”, are used interchangeably to describe the present invention. The word “tap” when used as a verb means to cut screw threads. A self-tapping device is one that cuts its own screw threads as it is turned. The terms “bone marrow” and “marrow” both mean the same thing.

In addition, the bore of the tap may also be referred to as the marrow extraction channel.

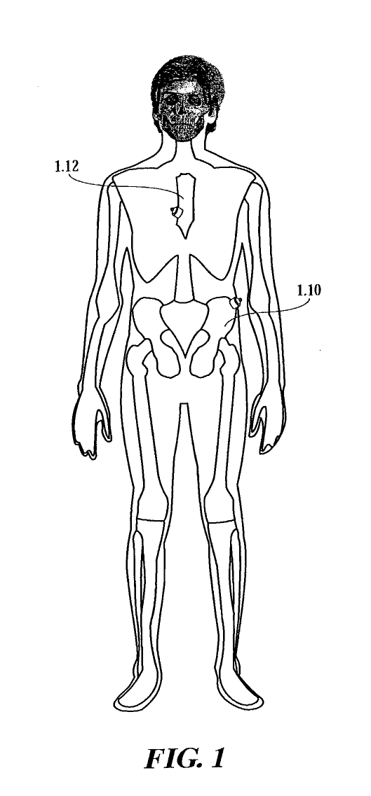

From FIG. 1, the dimensions and proportions of the invention and its elements may be varied to suit the site of implantation, such as the pelvis (hip bone 1.10) or sternum (breast bone 1.12) or whether it is to protrude from the surface of the skin. For sub-dermal implantation at the hip bone, the bore or marrow extraction channel is about 6 mm and the overall length of the tap is about 15 mm.

The surgical procedure to implant the device is briefly described as follows. A person skilled in the art will appreciate that variations of the technique described here may be used to achieve the same results.

One dose of antibiotics may be administered to the patient at induction of general anesthesia. The surgical site is disinfected and an incision made in the skin and fat layers are made.

For a sub-dermal implantation, a semi-circular incision in the fat layer is made. For a tap that protrudes from the surface of the skin, an incision perpendicular to the surface of the bone is made. This flap of tissue is reflected to expose the bone at the site.

A bone drill of diameter less than the external diameter of the device to be implanted, is used to drill through the outer cortex or hard thickness of the bone (also known as cortical or compact bone) to the interior of the bone (also known as spongy bone or bone marrow).

The site is then cleansed of bone fragments and tissue and the thickness of the cortical bone determined with a gauge.

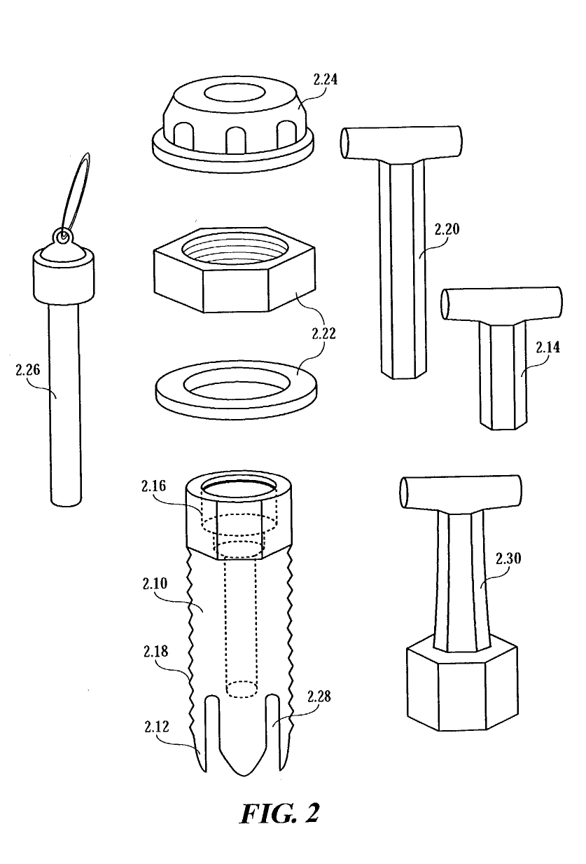

Referring to FIG. 2, a bone marrow tap 2.10, the present invention, of suitable length, diameter and thread characteristics (eg basic height of the thread, thread angle, pitch, etc) for the implantation site, is then selected.

The bone marrow tap has self-tapping threads 2.18 and its self-tapping end has recesses or slots 2.28 for the clearance of bone fragments as the self-tapping threads engage the hard cortical bone.

It is placed with its self-tapping end 2.12 onto the opening of the bone. A specially-designed wrench 2.14 with a shaft of square or polygonal cross-section, is mated with a corresponding recess 2.16 at the cap end of the device and turned to implant the device.

The self-tapping threads 2.18 of the device will seat the device into the hole as these self-tapping threads engage the thickness of the cortical bone.

As the wrench 2.14 mates with the cap end of the tap, its T-shaped handle affords the surgeon the means to generate sufficient trans-axial force to drive the tap into the bone and engage the threads of the tap with the cortical bone.

Once the tap is correctly seated, another wrench 2.20 with a longer shaft to drive the tap may be used to accommodate greater thickness of subcutaneous fat.

The desired depth of implantation is attained when the opening of the device just breaches the interior surface of cortical bone. The tip of the device should not protrude too much into the spongy bone.

One or more washer and locking nut sets 2.22 are installed onto the threaded exterior of the bone device to firmly anchor the device. The functions of the washer and locking nut sets are to prevent loosening of the multi-tap and to increase the area of the device in contact with bone so as to reduce forces to the bone.

Other variations to achieve the same function of the locking nut and washer sets are within the scope and spirit of the present invention. Some embodiments are described below. As an example, the cap may act as both cover and locking nut 4.10 to anchor the tap (FIG. 4).

Once the tap is implanted, a marrow sample may be taken.

The bore of the device is then closed with a removable sealing plug 2.26 inserted. Plugs of different lengths are available to suit the depth of implantation of the tap. The plug is designed such that it will not shift or move once it is applied. This may be achieved by means such as the plug resting on an internal shoulder of the bore at the cap end of the tap.

After the bore is plugged, the tap is capped and the surgical site or wound is then close in layers, cleansed with a suitable disinfectant and suitable dressings applied.

For subsequent repeated examinations of a tap that protrudes from the surface of the skin, all that would be needed is that the site be disinfected, the cap 2.24 and sealing plug 2.26 removed, and bone marrow extracted. Then the sealing plug is replaced and the device re-capped, the wound cleaned and suitable dressings applied.

Variations of the present invention will now be discussed, together with its distinguishing features from U.S. Pat. No. 5,120,312.

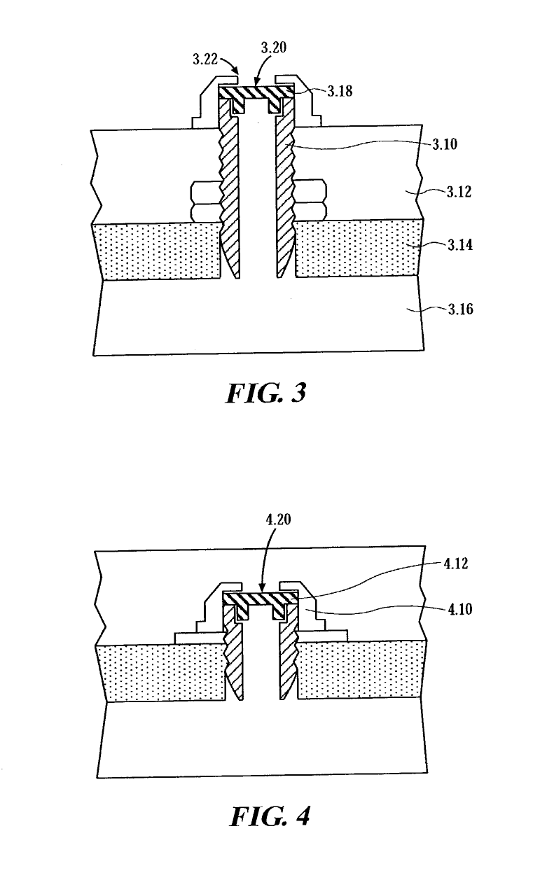

In another embodiment of the present invention, the plug may be in the form of a stopper which is not normally removed for repeated biopsies. Referring to FIG. 3, 3.12 is the layer of fat overlying the cortical bone layer 3.14 and the interior of the bone 3.16.

The plug (3.18 in FIG. 3 and 4.12 in FIG. 4) may be shortened and has a self-sealing septum (3.20 in FIG. 3 and 4.114 in FIG. 4). In conjunction with an aperture in the cap (3.22 in FIG. 3 and 4.16 in FIG. 4), the plug allows a needle to be introduced into the bore of the tap to access the interior of the bone.

If the tap does not protrude from the surface of the skin (such as the one shown in FIG. 4), the plug 4.10 with the self-sealing septum 4.10 may be used to allow a needle to puncture the skin, fat layer and septum to access the bore of the tap.

With this invention, pain and discomfort to the patient is minimized as repeated boring of cortical bone is avoided.

The parts of the invention may be made of one or more materials as long as the materials are hypoallergenic and compatible with biological tissue. Such materials must also have the suitable physical characteristics of strength and durability and are capable of being sterilized for surgical use. They should also be radio-opaque so that they may be seen under x-ray if the need arises.

Examples of such materials include 316L stainless steel, titanium alloys, and various engineering plastics, composites and ceramics.

Various advantages and distinguishing features of the present invention will now be discussed.

The device specifically designed to allow repeated access to bone marrow. Contents of the interior of the bone are retained by means of a removable plug 2.26 instead of a membrane, unlike that in U.S. Pat. No. 5,120,312.

The cap element 2.24 of the present invention allows quick, repeated direct access to the interior of bones. The wrench elements 2.14 and 2.20 of the present invention allow quick implantation unlike that taught in U.S. Pat. No. 5,120,312.

Depending on the thickness of the subcutaneous fat overlying the bone, wrenches with different shaft lengths may be used. By starting with a wrench with a shorter shaft, the surgeon has greater control in determining the insertion angle of the tap.

At this point in the surgical procedure, the advantage of having a small bevel at the lip of the recess 2.16 at the cap end of the tap will be apparent. As the tap is implanted below the level of the skin, the surgeon's view of tap may be partially obscured but the bevel will aid in the mating of subsequent wrenches.

The secure mating of the marrow tap and wrench, the short shaft and T-handle of the wrench all contribute in allowing the surgeon to implant the tap with careful, controllable turns of the wrench. As appropriate, the handle and shaft of the wrench may be in other configurations, such as in an L-shape arrangement, to suit various surgical requirements.

In another embodiment of the present invention, the wrench may be a socket wrench 2.30 (see also 6.30 in FIG. 6) that engages the exterior of the tap which has a corresponding exterior square or polygonal cross section.

The beveled tip of the marrow tap helps to center the tap upon seating with the hole in the bone. In the preferred embodiment, this bevel is about 15 degrees but this angle may be varied within the scope of the invention. The bevel also helps the threads to better grip the cortical bone at the start of the tapping procedure.

The recesses or slots at the tip allow any bone fragments to collect in the bore of the tap where they may be removed instead of allowing the fragments to clog the threads of the tap where they may compromise the secure implantation of the tap or complicate healing of the surgical site.

The bore of the tap is also sufficiently large to permit an appropriate instrument to be inserted to extract a sample of bone marrow. For the most common application of bone marrow examination, the bore has a diameter of 6 mm. At the same time, a variety of diameters and overall sizes for the present invention may be varied while remaining within the scope of the present invention. The choice of bore diameter and tap size is carefully selected to match the implantation site or medical circumstances.

The thickness of the cortical bone is variable between individuals and depth of implantation of the bone tap may be adjusted and then anchored by some means of preventing the tap from loosening such as one or more washer and locking nut sets 2.22 fastened along the exterior of the tap.

Conventional washers may be substituted with spring-loaded washers, compressible washers or other devices with the same function, such as Bellville type washers, may be used to prevent loosening of the tap and to cushion any impact against the cap end of the tap. Washers made of resilient materials with inherent springy or spongy properties may also be used.

FIG. 3 shows one embodiment of the present invention 3.10 that is implanted, anchored to the cortical bone 3.14, through the layer of fat 3.12 and protruding from the level of the skin. This embodiment has cap with an orifice, and a plug with a septum 3.20 for repeated access to the marrow.

FIG. 4 shows another embodiment of the device: one that does not protrude from the level of the skin. This embodiment has a cap and plug arrangement similar to that in FIG. 3.

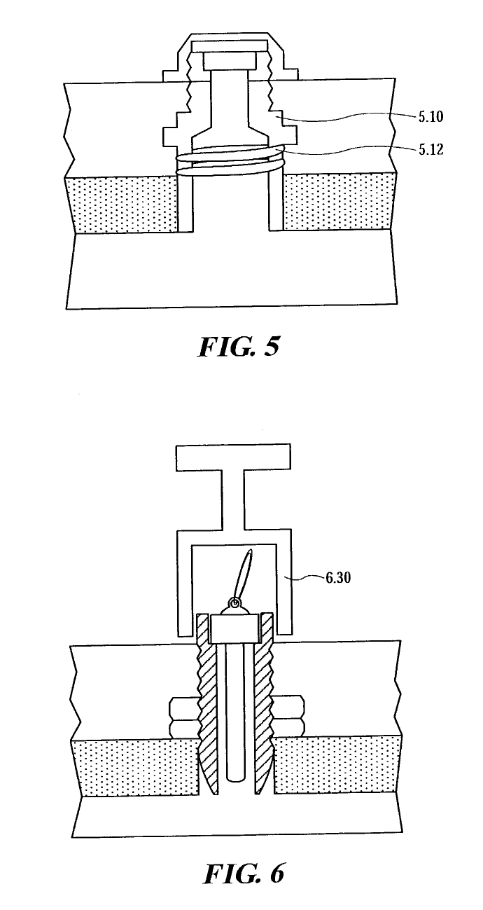

FIG. 5 is one embodiment with a flared base 5.10 in lieu of lock nuts. Here, washers with dampening capabilities (eg spring washers 5.12) are used such that the need for separate lock nut and washers is obviated.

Other alternative embodiments of some elements of the present invention are also possible and within the scope of the present invention.

The sealing plug may have, at the end nearest to the exterior (the cap end), a loop of hypoallergenic, biocompatible material to allow removal of the plug during subsequent biopsies. Alternatively, ridges, shoulders or knobs may be present to allow the sealing plug to be removed with the fingers or with surgical instruments like forceps or hemostats.

The sealing plug may be made of composite materials and it may be impregnated with medicaments such as antibiotics to reduce infection and to promote healing of the wound, or with heparin to prevent clotting of blood in the bore.

Likewise, various chemicals, biochemicals or pharmaceuticals may be incorporated into the materials or surfaces of the elements of the present invention, such as the plug, to promote healing, prevent or reduce infection or to obtain some desired medical outcome.

The cap may be of a square or polygonal (eg hexagonal) cross section to allow application or removal with a socket wrench. Alternatively, it may have a circular circumferential cross-section, the exterior of which may be ridged or knurled to provide purchase for fingers to tighten or loosen the cap. As mentioned above, it may have an orifice to allow a needle to puncture the septum of the plug beneath it to access the interior of the bone.

To further prevent leakage from the bore of the tap, the plug may have o-rings or flanges on its shaft where it mates with the cap. A ring wedge on the inside of the cap may be present to seal the cap against the opening of the tap.

The dimensions and shapes of the sealing plug and cap are chosen to prevent movement or shifting of the sealing plug within the bore of the marrow tap. An internal sleeve may be present on the underside of the cap that presses against the top of the sealing plug to prevent its movement.

A locator loop of biocompatible and radio-opaque material at exterior of the cap may be used to mark the site of the tap for subsequent biopsies. This is especially useful for taps that do not protrude above the surface of the skin.

As described above, for a tap that protrudes from the surface of the skin, repeated samples of marrow may be taken for examination by disinfecting the site, uncapping the tap and removing the plug. After the sample is taken, the original sealing plug or a new sterile plug may be used and the tap capped again.

For a tap that is implanted beneath the surface of the skin a cap and plug arrangement in which a needle may puncture the septum is used. With this arrangement, the tap is easily felt through the layer of fat and a hypodermic needle may be used to puncture through the fat layer and septum without the need to expose the tap. This arrangement is desirable to minimize infection during biopsies.

When the marrow tap is no longer needed, it may be removed.

To do this, the surgical site is re-exposed by making an incision over the previous incision's scar. The cap is re-moved and the lock nut, washers and tap unscrewed and removed. The orifice in the bone may be plugged with bone wax and any hemostasis controlled. Tissue fascia is replaced and closed in the usual manner. The wound is cleansed with a suitable disinfectant and dressings are applied.

It will be appreciated that although one preferred embodiment has been described in detail, various modifications and improvements can be made by a person skilled in the art without departing from the scope of the present invention.

What is claimed is:

1. A device reversibly implanted in bones that allows repeated access to the interior of said bones without the need to repeatedly bore holes in bone each time access is desired.

2. A device reversibly implanted in bones, said device further comprising:
a rigid tube;
a means of engaging said tube with bone;
a means of turning said tube;
a means of anchoring said tube to bone;
a means of reversibly sealing bore of said tube and
a means reversibly capping one end of said tube.

3. A device according to claim 2, said rigid tube further comprising a a tip end and a cap end;

4. A device according to claim 2, said means of engaging said tube with bone further comprises self-tapping threads on external surface of said tube.

5. A device according to claim 2, said means of turning said tube further comprises at least one wrench and means of engaging said at least one wrench.

6. A device according to claim 2, said means of anchoring said tube to bone further comprises said self-tapping threads and means of preventing said tube from loosening from said bone.

7. A device according to claim 2, said means of reversibly sealing bore of said tube further comprises a plug seated in bore of said tube such that said plug does not move within bore after said plug is inserted.

8. A device according to claim 2, said means of reversibly capping one end of tube further comprises a screw-on cap.

9. A device according to claim 3, said tip end further comprises a bevel and at least one recess.

10. A device according to claim 5, said means of engaging said at least one wrench further comprises a recess accepting said wrench.

11. A device according to claim 5, said means of engaging said at least one wrench further comprises a recess in said at least one wrench accepting said cap end of said device.

12. A device according to claim 6, said means of preventing said tube from loosening further comprises at least one washer and at least one lock nut.

13. A device according to claim 12, said at least one washer comprising washer of compressive nature.

14. A device according to claim 7, said plug further comprising ridges, shoulders, knobs, o-rings, flanges and septa.

15. A device according to claim 2, elements of said device incorporating at least one chemical to obtain at least one desirable medical outcome.
